(19) United States
(12) Patent Application Publication
(10) Pub. No.: US 2010/0281565 A1
(43) Pub. Date: Nov. 4, 2010

(54) Transformation of Crambe Abyssinica

(75) Inventors: Dean Engler, Moraga, CA (US); Belen Montanez, Scottsdale, AZ (US)

Correspondence Address: MENDEL 2 C/O MOFO SF, 425 MARKET STREET, SAN FRANCISCO, CA, 94105, US

(73) Assignee: MENDEL BIOTECHNOLOGY, INC., Haward, CA (US)

(21) Appl. No.: 12/742,745

(22) PCT Filed: Nov. 17, 2008
(86) PCT No.: PCT/US2008/083752

§ 371 Date: May 13, 2010

Related U.S. Application Data
(60) Provisional application No. 61/004,109, filed on Nov. 21, 2007.

(51) Int. Cl. A01H 5/00; A01H 4/00; C12N 15/82

(52) U.S. Cl. 800/268; 800/306; 800/278

(57) ABSTRACT

The present invention provides methods for transforming Crambe species plants by producing embryogenic callus or somatic tissue, which is transformed, selected and regenerated into whole transgenic plants. The invention also pertains to a plant of the genus Crambe, and a method for producing the plant, where the plant has greater hypocotyl regenerability than a control plant.

### FIELD OF THE INVENTION

The present invention relates methods for genetically altering cells of higher plants and obtaining regenerated plants from said cells.

### BACKGROUND OF THE INVENTION

Crambe abyssinica (also known as Abyssinian mustard, Abyssinian kale, colewart, and datran) is a plant species that is a source of a vegetable oil generally used for industrial purposes. Vegetable-derived industrial oils are desirable for several reasons, among these are high biodegradability and low environmental toxicity. Erucic acid confers to vegetable industrial oils several desirable characteristics, including enhanced lubricity, enhanced wettability, low reactivity, high smoke point, and oxidative stability. Erucic acid-rich oil is an excellent mold lubricant for continuous steel casting. Uses for high erucic acid (HEA) oils also include their use in detergents, as polymer additives, in hydraulic fluids, quenchants, personal care products, cosmetics, and surfactants. In addition, HEA oils are used in the production of paints and coatings, in the manufacturing of nylon, plastics, and hard waxes, and as alternative fuels.

Erucic acid-rich oil may be obtained by crushing seeds of Crambe abyssinica or high erucic acid rapeseed (HEAR). Crambe abyssinica has several advantages over HEAR. For example, C. abyssinica is not a Brassica species, and does not cross with Brassica napus (rapeseed), the source of canola oil for human consumption. Crambe abyssinica is not susceptible to many of the pests and diseases that adversely affect Brassica. Crambe abyssinica is also more drought tolerant, more immune to lodging, and less susceptible to weed problems than brassicates. For these reasons and others, Crambe abyssinica is the cheapest source of erucic acid.

Crambe abyssinica has been shown to be amenable to tissue culture techniques (Jones, 1988) and Crambe plants may be regenerated from single cell culture (Gao, 1998, Sonntag and Gramenz, 2004, and U.S. Pat. No. 4,665,031 to Peron). Genetically modified Crambe for the production of hydroxylated fatty acids has been proposed (U.S. Pat. No. 6,936,728 to Somerville et al.), and transformation of Crambe abyssinica has been described in a preliminary report using an Agrobacterium-based approach (Sonntag, 2001). In this very brief report, Sonntag et al, 2001, partially described a method for production of transformed plants from cocultivated cotyledon explants of the Crambe abyssinica variety Galactica. They described mixing the explants with Agrobacterium, but do not indicate what the concentration of the bacterial cells should be. They described the medium that they used for cocultivation of the cotyledons, but they did not indicate how long the explants were left on this medium prior to transfer, nor any of the environmental conditions of the cocultivation period (i.e. temperature, lighting, photoperiod and the like). Following the cocultivation step, they described incubation of the explants on a shoot induction medium, but again did not describe any of the environmental conditions which were used for shoot induction. They did not indicate if the explants were transferred to fresh media at any point in the process other than the transfer from cocultivation medium to shoot induction medium. Knowledge of the above parameters is critical for successful plant transformation. Sonntag et al (2001) attempted to transform 2 varieties other than Galactica (Carmen and Bel Ann) with this method, but these attempts failed. The described method was also extremely inefficient as indicated by the fact that of the 153 Galactica regenerated shoots resulting from the method, only three were claimed to be transgenic, with 150 having “escaped” from the selection (98.3% escapes; an “escape” may be defined as a non-transformed shoot that forms during and in spite of a selection process, i.e., the shoot, while lacking the appropriate selection marker, is not limited in its formation by the selection process, which may be inadequate, and hence escapes the selection step).

Improvement of the characteristics and yield of industrial vegetable oils may be obtained with the use of genetic engineering techniques. For example, it may be possible to increase the value of Crambe abyssinica oil by genetically modifying the plant to produce enzymes in the developing embryos during seed formation which cause the seed storage lipids to be partly in the form of liquid waxes rather than triglycerides. Liquid waxes currently have a high value as an ingredient in cosmetics, and would be expected to confer increased heat and pressure stability on seed storage lipids (see Lassner et al., 1999).

Genetic engineering of Crambe abyssinica involves introduction of exogenous DNA into Crambe abyssinica cells and the regeneration of said transformed cells into whole C. abyssinica plants. These techniques for gene introduction are preferably efficient in all steps of the process, from DNA delivery into the plant cells to regeneration of intact plants from the transformed cells.

Embryogenic callus is a generally useful tissue for the purpose of producing whole transformed plants. In other plant species, embryogenic callus has generally been found to be efficiently transformable, regenerable into whole plants, and these two processes can be separated into two separate steps. That is, a transformation protocol applied to embryogenic callus will allow the production of transformed embryogenic callus, and the transformed embryogenic callus can be amplified and regenerated into whole plants in a second step. The ability to separate the transformation and regeneration steps avoids the need to regenerate the few cells that are initially transformed, and therefore leads to efficient transformation protocols. A second way to separate the transformation and regeneration steps is to produce transformed undifferentiated callus, grow that callus, and then regenerated whole plants by organogenesis.

Production of embryogenic callus is often difficult, genotype-specific, and time-consuming. Protocols that employ this tissue are therefore typically difficult to develop and time-consuming to follow. These protocols also typically suffer from the lack of genotype independence. Despite these limitations, we investigated the possibility of the use of embryogenic callus for Crambe transformation, and were surprised by our observation that in Crambe the production of embryogenic callus was rapid and not limited to particular genotypes. We investigated three genotypes; Meyer, Bel Ann, and a wild Crambe accession, and we produced embryogenic callus from all three.

### SUMMARY OF THE INVENTION

The present invention is directed to methods of producing transformed Crambe abyssinica plants, in particular by transforming cells present in hypocotyl explants, or embryogenic callus and obtaining regenerated plants therefrom. The method of the invention comprises two alternative pathways, each ultimately arriving at a transformed Crambe abyssinica plant. In one method, hypocotyl explants are transformed and callus is produced which is then regenerated into whole plants. Alternatively, source tissue is cultured to produce somatic embryo or pro-embryo structures. These are cultured to produce embryogenic callus, which is in turn transformed to produce transformed embryogenic callus. The transformed embryogenic callus is then cultured to produce transformed regenerated plants.

The transformation methods preferably include introduction of a marker to permit selection or screening of transformed cells. Transformed callus or transformed embryogenic callus may be cultured to multiply or increase the amount of transformed callus or embryogenic callus. Subsequently, germination or regeneration is carried out to produce mature plantlets which may be transferred to soil conditions.

In one embodiment of this invention, the method comprises: (a) culturing somatic Crambe abyssinica plant tissue to obtain embryogenic material; (b) genetically transforming the embryogenic material produced in step (a) by cocultivating with Agrobacterium cells carrying exogenous DNA sequence(s), the DNA sequence(s) which typically includes a selectable marker gene as well as one or more genes of interest to be expressed; (c) multiplying the transformed somatic embryo culture to produce additional transformed somatic embryos; and (d) germinating the transformed somatic embryos to produce a mature plantlet capable of being transferred to soil conditions.

In another embodiment of this invention, the method comprises: (a) genetically transforming Crambe abyssinica plant tissue by cocultivating with Agrobacterium cells carrying exogenous DNA sequence(s), the DNA sequence(s) which typically includes a selectable marker gene as well as one or more genes of interest to be expressed; (b) culturing the transformed callus; (c) inducing organogenic regeneration of the transformed callus to produce a mature plantlet capable of being transferred to soil conditions.

In yet a third embodiment of this invention, the method includes the use of particle gun transformation. Crambe abyssinica cells are grown in tissue culture, the cells bombarded with polynucleotide-coated particles with the particle gun, transgenic cells are selected on selection media, and the transgenic cells are regenerated into transformed plants.

A variety of traits, including agronomic traits such as disease resistance or yield, and traits that improve quality such as improved oil compositions, may be stably introduced into Crambe abyssinica using the methods of the invention.

The invention is also directed to a method for producing a plant of the genus Crambe that has greater hypocotyl regenerability than a control plant (for example, a wild-type Crambe plant of the same species, or a parental line from which the plant that has greater hypocotyl regenerability is derived). The method steps include first germinating a seed of the genus Crambe, then removing a hypocotyl from the germinating seed and separating the hypocotyl into segments. The segments are transferred to a medium and an environment that supports hypocotyl segment regeneration, and the segments are incubated for a period of time sufficient for the segments to produce shoots. A specific segment that produces a higher number of shoots than the average number of shoots produced by all of the segments is selected, and preferably the specific shoot produces the highest number of shoots produced by all of the segments. An apical meristem from the shoot grown from the specific selected segment is then grown into a mature plant. The mature plant is then either self-pollinated, or pollinated with pollen of another plant that has been selected for greater hypocotyl regenerability than the control plant. A resulting progeny seed is collected, and a progeny plant is grown from the progeny seed, the progeny plant having greater hypocotyl regenerability than the control plant.

### DETAILED DESCRIPTION

The present invention is directed to methods of genetically transforming Crambe abyssinica by selectively introducing exogenous DNA sequence(s) in order to obtain genetically altered C. abyssinica cells, in-vitro tissues, and plants. The methods involve use of somatic C. abyssinica plant tissue, embryogenic material, seedling tissue, mature plant tissue, DNA sequence(s) to be introduced, Agrobacterium cells to carry DNA sequence(s) and mediate their transfer to C. abyssinica cells, and culture media suitable for the various steps, including embryogenic callus induction, embryo proliferation, and embryo and plantlet regeneration, as described.

Throughout this disclosure, various information sources are referred to and/or are specifically incorporated. The information sources include scientific journal articles, patent documents, textbooks, and World Wide Web browser-inactive page addresses. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the invention.

As used herein and in the appended claims, the singular forms “a”, “an”, and “the” include the plural reference unless the context clearly dictates otherwise. Thus, for example, a reference to “a host cell” includes a plurality of such host cells, and a reference to “a stress” is a reference to one or more stresses and equivalents thereof known to those skilled in the art, and so forth.

### DEFINITIONS

“Polynucleotide” is a nucleic acid molecule comprising a plurality of polymerized nucleotides, e.g., at least about 15 consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single-stranded.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a nucleic acid construct, or otherwise recombined with one or more additional nucleic acid.

An “isolated polynucleotide” is a polynucleotide, whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as chemical modification or folding to obtain a functional protein or polypeptide. A gene may be isolated, partially isolated, or found with an organism's genome.

Operationally, genes may be defined by the cis-trans test, a genetic test that determines whether two mutations occur in the same gene and that may be used to determine the limits of the genetically active unit (Rieger et al., 1976). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “polypeptide” is an amino acid sequence comprising a plurality of consecutive polymerized amino acid residues e.g., at least about 15 consecutive polymerized amino acid residues. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

“Portion”, as used herein, refers to any part of a protein used for any purpose, but especially for the screening of a library of molecules which specifically bind to that portion or for the production of antibodies.

“Complementary” refers to the natural hydrogen bonding by base pairing between purines and pyrimidines. For example, the sequence A-C-G-T (5′->3′) forms hydrogen bonds with its complements A-C-G-T (5′->3′) or A-C-G-U (5′->3′). Two single-stranded molecules may be considered partially complementary, if only some of the nucleotides bond, or “completely complementary” if all of the nucleotides bond. The degree of complementarity between nucleic acid strands affects the efficiency and strength of hybridization and amplification reactions. “Fully complementary” refers to the case where bonding occurs between every base pair and its complement in a pair of sequences, and the two sequences have the same number of nucleotides.

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae (see for example, Daly et al., 2001, Ku et al., 2000; and also Tudge, 2000).

A “control plant” as used in the present invention refers to a plant cell, seed, plant component, plant tissue, plant organ or whole plant used to compare against transformed, transgenic or genetically modified plant for the purpose of identifying an enhanced phenotype in the transformed, transgenic or genetically modified plant. A control plant may in some cases be a transformed or transgenic plant line that comprises an empty vector or marker gene, but does not contain the recombinant polynucleotide of the present invention that is expressed in the transformed, transgenic or genetically modified plant being evaluated. In general, a control plant is a plant of the same line or variety as the transformed, transgenic or genetically modified plant being tested. A suitable control plant would include a genetically unaltered or non-transgenic plant of the parental line used to generate a transformed or transgenic plant herein.

“Transformation” refers to the transfer of a foreign polynucleotide sequence into the genome of a host organism such as that of a plant or plant cell. Typically, the foreign genetic material has been introduced into the plant by human manipulation, but any method can be used as one of skill in the art recognizes. Examples of methods of plant transformation include Agrobacterium-mediated transformation (De Blaere et al., 1987) and biolistic methodology (Klein et al, 1987).

A “transformed plant”, which may also be referred to as a “transgenic plant” or “transformant”, generally refers to a plant, a plant cell, plant tissue, seed or calli that has been through, or is derived from a plant that has been through, a transformation process in which a nucleic acid construct that contains at least one foreign polynucleotide sequence is introduced into the plant. The nucleic acid construct, which may be an expression vector or expression cassette, a plasmid, or a DNA preparation, contains genetic material that is not found in a wild-type plant of the same species, variety or cultivar. The genetic material may include a regulatory element, a transgene (for example, a foreign sequence derived from another plant line or species), an insertional mutagenesis event (such as by transposon or T-DNA insertional mutagenesis), an activation tagging sequence, a mutated sequence, a homologous recombination event or a sequence modified by chimeraplasty. In some embodiments the sequence may be derived from the host plant, but by their incorporation into an expression vector of cassette, represent an arrangement of the polynucleotide sequences not found a wild-type plant of the same species, variety or cultivar. Such an expression cassette is provided with a plurality of restriction sites for insertion of the gene of interest sequence to be under the transcriptional regulation of the regulatory regions. The expression cassette may additionally contain identification or selectable marker genes.

A nucleic acid construct such as a plasmid, an expression vector or expression cassette typically comprises a polypeptide-encoding sequence operably linked (i.e., under regulatory control of) to appropriate inducible or constitutive regulatory sequences that allow for the controlled expression of polypeptide. The expression vector or cassette can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell. The term “nucleic acid construct” is not intended to limit the present invention to nucleotide constructs comprising DNA. Nucleic acid constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs which can be employed in the methods of the present invention for transforming Crambe abyssinica plants including, but not limited to, those comprised of deoxyribonucleotides, ribonucleotides and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures and the like.

Methods of the invention may employ a nucleotide construct that is capable of directing, in a transformed plant, the expression of at least one protein, or at least one RNA, such as, for example, an rRNA, a tRNA and an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods of the invention may employ a nucleotide construct that is not capable of directing, in a transformed plant, the expression of a protein or an RNA.

An “untransformed plant” is a plant that has not been through the transformation process.

A “stably transformed” plant, plant cell or plant tissue has generally been selected and regenerated on a selection media following transformation.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a polypeptide's expression is altered, e.g., in that it has been knocked out, overexpressed, or ectopically expressed.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deficit or particular salt or sugar concentrations, or by the observation of the expression level of a gene or genes, e.g., by employing Northern analysis, RT-PCR, microarray gene expression assays, or reporter gene expression systems, or by agricultural observations such as increased cold or water deficit tolerance or an increased yield. Any technique can be used to measure the amount of, comparative level of, or difference in any selected chemical compound or macromolecule in the transformed or transgenic plants, however.

“Yield” or “plant yield” refers to increased plant growth, increased crop growth, increased biomass, and/or increased plant product production (e.g., plant oil or erucic acid), and is dependent to some extent on temperature, plant size, organ size, planting density, light, water and nutrient availability, and how the plant copes with various stresses, such as through temperature acclimation and water or nutrient use efficiency.

“Somatic embryo” refers to a structure similar to a zygotic embryo which arises from a somatic cell. Somatic embryos can germinate and form whole plants which become “clones” of the source plant. In other words, the whole plants that germinate from the somatic embryos have a genetic make-up that is identical to the source plants.

“Embryogenic callus” refers to cells that are capable of becoming somatic embryos. These cells are usually produced by culture of different organs in vitro. Embryogenic callus may contain organized structures which are capable of maturing into somatic embryos.

“Nutrient media” typically comprises salts, a carbon source and vitamins at concentrations necessary to effect the maintenance of cultured plant cells.

In this description, “effective amount” refers to an amount of a given component necessary to effect the recited step.

### Vectors, Promoters, and Expression Systems

The present invention includes plants and methods for producing the plants, where the plants are transformed with recombinant nucleic acid constructs comprising one or more nucleic acid sequences. The constructs typically comprise a vector, such as a plasmid, a cosmid, a phage, a virus (e.g., a plant virus), a bacterial artificial chromosome (BAC), a yeast artificial chromosome (YAC), or the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors, cassettes and promoters are known to those of skill in the art, and are commercially available.

General texts that describe molecular biological techniques useful herein, including the use and production of vectors, cassettes and promoters and many other relevant topics, include Berger and Kimmel, 1987, Sambrook et al., 1989, and Ausubel, 1997-2001. Any of the identified sequences can be incorporated into a cassette or vector, e.g., for expression in plants. A number of expression vectors or cassettes suitable for stable transformation of plant cells or for the establishment of transgenic plants have been described including those described in Weissbach and Weissbach, 1989, and Gelvin et al., 1990. Specific examples include those derived from a Ti plasmid of Agrobacterium tumefaciens, as well as those disclosed by Herrera-Estrella et al., 1983, Bevan 1984, Klee 1985, for dicotyledonous plants.

Typically, plant transformation vectors include one or more cloned plant coding sequence (genomic or cDNA) under the transcriptional control of 5′ and 3′ regulatory sequences and a dominant selectable marker. Such plant transformation vectors typically also contain a promoter (e.g., a regulatory region controlling inducible or constitutive, environmentally- or developmentally-regulated, or cell- or tissue-specific expression), a transcription initiation start site, an RNA processing signal (such as intron splice sites), a transcription termination site, and/or a polyadenylation signal.

Examples of constitutive plant promoters which can be useful for expressing the TF sequence include: the cauliflower mosaic virus (CaMV) 35S promoter, which confers constitutive, high-level expression in most plant tissues (see, e.g., Odell et al., 1985); the nopaline synthase promoter (An et al., 1988); and the octopine synthase promoter (Fromm et al., 1989).

A variety of plant gene promoters that regulate gene expression in response to environmental, hormonal, chemical, developmental signals, and in a tissue-active manner can be used for expression of a TF sequence in plants. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc), timing, developmental stage, and the like. Numerous known promoters have been characterized and can favorably be employed to promote expression of a polynucleotide of the invention in a transgenic plant or cell of interest. For example, tissue specific promoters include: seed-specific promoters (such as the napin, phaseolin or DC3 promoter described in U.S. Pat. No. 5,773,697 to Tomes, 1998), fruit-specific promoters that are active during fruit ripening (such as the dru 1 promoter (U.S. Pat. No. 5,783,393 to Kellogg, 1998), or the 2A11 promoter (U.S. Pat. No. 4,943,674 to Houck and Pear, 1990) and the tomato polygalacturonase promoter (Bird et al., 1988), root-specific promoters, such as those disclosed in U.S. Pat. No. 5,618,988 to Hauptmann, et al., 1997, U.S. Pat. No. 5,837,848 to Ely, 1998, and U.S. Pat. No. 5,905,186 to Thomas et al., 1999, pollen-active promoters such as PTA29, PTA26 and PTA13 (U.S. Pat. No. 5,792,929 to Mariani, 1998), promoters active in vascular tissue (Ringli and Keller, 1998), flower-specific (Kaiser et al., 1995), pollen (Baerson et al., 1994), carpels (Ohl et al., 1990), pollen and ovules (Baerson et al., 1993), auxin-inducible promoters (such as that described in van der Kop et al., 1999 or Baumann et al., 1999), cytokinin-inducible promoter (Guevara-Garcia, 1998), promoters responsive to gibberellin (Shi et al., 1998, Willmott et al., 1998) and the like. Additional promoters are those that elicit expression in response to heat (Ainley et al., 1993), light (e.g., the pea rbcS-3A promoter, Kuhlemeier et al., 1989, and the maize rbcS promoter, Schaffner and Sheen, 1991); wounding (e.g., wunl, Siebertz et al., 1989); pathogens (such as the PR-1 promoter described in Buchel et al., 1999, and the PDF1.2 promoter described in Manners et al., 1998), and chemicals such as methyl jasmonate or salicylic acid (Gatz, 1997). In addition, the timing of the expression can be controlled by using promoters such as those acting at senescence (Gan and Amasino, 1995); or late seed development (Odell et al., 1994).

Plant expression vectors or cassettes can also include RNA processing signals that can be positioned within, upstream or downstream of the coding sequence. In addition, the expression vectors can include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

Production of Stably Transformed Crambe abyssinica Plants

Methods of the invention involve producing a stably transformed Crambe abyssinica plant. Generally, a transformed Crambe abyssinica plant of the invention is a capable of producing at least one progeny plant and preferably, at least one transformed progeny plant.

This invention is directed to methods of genetically transforming Crambe abyssinica by selectively introducing exogenous DNA sequence(s) in order to obtain genetically altered Crambe abyssinica cells, in-vitro tissues, and plants. This invention is useful with all Crambe abyssinica plants, including commercial and pre-commercial varieties, breeding lines, and wild varieties. This invention is also efficient in that it results in few (for example, less than 10%) escapes, and many transformed plants can be produced by a single operator doing one experiment.

The methods of this invention may involve use of somatic Crambe abyssinica plant tissue, embryogenic material, seedling tissue, mature plant tissue, DNA sequence(s) to be introduced, Agrobacterium cells to carry DNA sequence(s) and mediate their transfer to Crambe abyssinica cells, and culture media suitable for the various steps, including embryogenic callus induction, embryo proliferation, and embryo and plantlet regeneration, as described. Any plant culture medium known in the art may be employed in the methods of the invention including, but not limited to, a transformation support medium, an identification or selection medium and a regeneration medium. Typically, such media comprise water, a basal salt mixture and a carbon source, and may additionally comprise one or more other components known in the art, including but not limited to, vitamins, co-factors, myo-inositol, selection agents, charcoal, amino acids, silver nitrate and phytohormones. If a solid plant culture medium is desired, then the medium additionally comprises a gelling agent such as, for example, gelrite, agar or agarose.

The Crambe abyssinica seeds for use with this invention may be purchased from suppliers of cultivated seeds, and may be of any cultivated variety such as the variety Meyer, or Bel Ann, or Galactica, or may be collected from wild or non-cultivated plants from nature, or may be breeding lines that are held by breeders of Crambe abyssinica.

Any Crambe abyssinica plant tissue, including mature and immature somatic plant tissue, can be used as a source of explant material in the present invention as long as it is capable of producing embryogenic material or undifferentiated callus which is capable of regeneration. Suitable somatic plant tissue includes tissue from immature flowers, seedling tissue such as hypocotyl cylinders or hypocotyl disks, cotyledons, and roots, and mature plant tissues such as leaves, stems, flowering stalks, and the like Immature flowers and hypocotyl disks cut from tissue close to the apical meristem are the preferred somatic plant tissue sources.

The present invention was practiced with at least two protocols. These included:
(1) Embryogenic callus was produced, the callus was subjected to a transformation protocol, transformed embryogenic callus was selected and the transformed embryogenic callus was germinated or regenerated into whole transgenic plants. Using this method, transformed rooted shoots have been produced which have been grown into mature plants. The seeds resulting from self-pollination of one of these plants have been collected, germinated, and assayed for the presence of the GUS transgene. Seventeen of the twenty three seedlings assayed expressed the GUS transgene as indicated by dark blue staining These data fit a Mendelian segregation ratio of 3 GUS positive: 1 GUS negative expected from a non-chimeric transformed plant with the transgene stably integrated at a single locus, and heritable through gametes from one generation to the next.
(2) Somatic tissue was subjected to a transformation protocol, transformed undifferentiated callus was selected and the transformed undifferentiated callus was regenerated by organogenesis into whole transgenic plants. Using this method, transformed shoots have been produced.

### Transformation of Crambe Embryogenic Callus

In the first method, the preferred source material for the production of embryogenic callus was flower tissue, and in particular tissue from immature flowers. The preferred explants for induction of embryogenic callus were the immature stamens, immature pistils, and immature petals. Most preferred were the immature stamens. Clusters of immature flowers were collected from Crambe abyssinica plants, preferably before the flowers in the cluster have opened. The clusters of immature flower buds were sterilized, preferably by immersion into a solution of bleach followed by rinsing with sterile distilled water. Flower buds which were 0.5 to 1.5 mm in diameter, preferably 1.0 mm in diameter were selected for dissection. Stamens which were minimally damaged or preferably undamaged were removed from the flower buds and transferred to solidified ECIGM medium. Plates with 25 to 50 of these stamens were incubated at 20° C. to 30° C., preferably 25° C. in light or dark conditions, and preferably in light conditions with 12 hour photoperiods. These conditions were maintained for two to four months with or without periodic transfer of the stamens to fresh ECIGM medium, preferably with transfer every three weeks, until embryogenic callus was apparent on some of the stamens. The embryogenic callus was then cultured in the same way and on the same medium as was used for its initiation, as described above. Clumps of embryogenic callus were broken into small pieces, generally about 1 mm in diameter at each transfer. The embryogenic calli were subcultured for a period of time necessary to increase their numbers to a desired level. Subculturing allowed the continuing maintenance of the somatic embryos as a source of starting materials. The embryogenic calli thus produced were used as targets for transformation.

In order to achieve the desired transformation, cocultivation with an Agrobacterium species carrying the exogenous DNA sequence to be transferred has been performed, and preferably is performed with an Agrobacterium species carrying the exogenous DNA sequence to be transferred. Alternatively, the embryogenic callus may be subjected to a transformation protocol such as particle gun DNA delivery.

For transformation by Agrobacterium cocultivation, incubation was achieved in a cocultivation medium which includes nutrients, an energy source, and an induction compound which was used to induce the virulence (vir) region of Agrobacterium to enhance transformation efficiency (transformation efficiency may be defined as the number of transformed shoots that are formed on selection divided by the number of explants used). The embryogenic callus and Agrobacterium cells could be placed on a filter paper matrix, such as Whatman #1, or glass microfibre filter, on the cocultivation medium during the cocultivation process. The induction compound could be any phenolic compound which is known to induce such virulence, preferably being acteosyringone (AS) present at from about 10 to 600 μM, and preferably at about 100-300 μM. Embryogenic calli were combined with the Agrobacterium cells in the cocultivation medium at a temperature typically in the range from about 20° C. to 28° C., generally for about three days. Calli may also be combined with Agrobacterium cells at a temperature that is preferably at about 22° C.-25° C., from two to four days, and generally for about three days. The medium was preferably kept in the dark and the cocultivation continued until the Agrobacteria grew to a level of observable bacterial growth. The Agrobacterium cells were initially present at a concentration form about 10⁷ to 10⁹ cells/ml, preferably at about 10⁸ cells/ml. Usually, a total of about 0.25 to 5 grams of embryogenic callus was used in a total culture volume of about 1 to 25 ml.

After transformation was completed, the transformed embryogenic callus was placed on a suitable selection medium that included a plant selection agent which permitted identification of transformed embryogenic callus based on the presence of the marker introduced as part of the exogenous DNA.

The transformed cells may be identified or selected and, if desired, regenerated into transformed plants. The methods of the invention do not depend on any particular method for identifying or selecting transformed cells from embryogenic callus and for regenerating such cells into transformed Crambe plants. Identification methods may involve utilizing a marker gene, such as green fluorescent protein (GFP), or a cell cycle gene such as CKI, Cyclin D. Methods for using GFP and cell cycle genes are found in U.S. Pat. Nos. 6,300,543, 60/246,349 and Ser. No. 09/398,858 and are incorporated by reference. Selection methods typically involve placing the embryogenic callus on a medium that contains a selective agent, promotes regeneration or both. If, for example, the nucleotide construct comprises a selectable marker gene for herbicide resistance that is operably linked to a promoter that drives expression in a plant cell, then selection of the transformed cells may be achieved by adding an effective amount of the herbicide to the medium to inhibit the growth of or kill non-transformed cells. Such selectable marker genes and methods of use are well known in the art. Methods and media employed in the regeneration of transformed Crambe plants from transformed cells of embryogenic callus are described herein. Generally, such methods comprise contacting Crambe embryogenic callus with a medium lacking phytohormones. Any method known in the art for identifying or selecting transformed plant cells and regenerating transformed Crambe plants may be employed in the methods of the present invention. This paragraph generally cites U.S. Pat. No. 7,057,089 to Ranch and Marsh, herein incorporated by reference.

The methods of the invention do not depend on a particular nucleotide construct. Any nucleotide construct that may be introduced into a plant cell may be employed in the methods of the invention. Nucleotide constructs of the invention comprise at least one nucleotide sequence of interest operably linked to a promoter that drives expression in a plant cell. The nucleotide constructs may also comprise identification or selectable marker gene constructs in addition to the nucleotide sequence of interest.

Selectable marker genes may be utilized for the selection of transformed cells or tissues. Selectable marker genes may also be utilized, and include, but are not limited to, GFP (see, for example, PCT patent publication WO9741228), genes encoding antibiotic resistance, such as nptII which encodes neomycin phosphotransferase II (NEO), hpt which encodes hygromycin phosphotransferase (HPT), and the monocot-optimized cyanamide hydratase gene (moCAH) (see, for example, U.S. Pat. No. 6,096,947) as well as genes conferring resistance to herbicidal compounds, such as glufosinate ammonium, bromoxynil, imidazolinones, and 2,4-dichlorophenoxyacetate (2,4-D). Also see generally, Yarranton (1992); Christopherson et al. (1992); Yao et al. (1992); Reznikoff (1992); Barkley et al. (1980); Hu et al. (1987); Brown et al. (1987); Figge et al. (1988); Deuschle et al. (1989); Fuerst et al. (1989); Deuschle et al. (1990); Gossen (1993); Reines et al. (1993); Labow et al. (1990); Zambretti et al. (1992); Bairn et al. (1991); Wyborski et al. (1991); Hillenand-Wissman (1989); Degenkolb et al. (1991); Kleinschnidt et al. (1988); Bonin (1993); Gossen et al. (1992); Oliva et al. (1992); Hlavka et al. (1985); Gill et al. (1988). This paragraph generally cites U.S. Pat. No. 7,057,089 to Ranch and Marsh. Such disclosures are herein incorporated by reference.

A selective media was used with portions of the calli, the latter typically being a mass in the range of about 100-200 mg each. The selection medium was a general growth medium, such as the proliferation medium ECIGM, described herein, supplemented with the selection agent. When cocultivation with Agrobacterium was used as a transformation method, the selective medium usually included an anti-Agrobacterium antibiotic capable of killing Agrobacterium without harming plant tissues, such as, for example, carbenicillin (100 to 500 mg/L), cefotaxime (100 to 500 mg/L), or timenton (50 to 250 mg/L; 150 mg/L is a preferred concentration). Suitable plant selection agents include, for example, Geneticin® ((2R,3S,4R,5R,6S)-5-amino-6-[(1R,2S,3S,4R,6S)-4,6-diamino-3-[(2R,3R,4R,5R)-3,5-dihydroxy-5-methyl-4-methylaminooxan-2-yl]oxy-2-hydroxycyclohexyl]oxy-2-(1-hydroxyethyl)oxane-3,4-diol; 1-100 mg/L; 25 mg/L is preferred), Asulam (2-200 mg/L), or kanamycin (50-500 mg/L).

It may be desirable to confirm transformation using a standard assay procedure such as Southern blotting, Northern blotting, restriction enzyme digestion, polymerase chain reaction (PCR) assays, or through the use of reporter genes. Suitable reporter genes and assays include glucuronidase (GUS) assays as described by Jefferson, GUS Gene Fusion Systems User's Manual, Cambridge, England (1987) and GFP expression. These assays can be performed immediately following the transformation procedures or at any subsequent point during the regeneration of the transformed plant materials according to the present invention.

The transformed embryogenic calli were placed on a regeneration medium, typically one that included plant selection agents in order to produce fully transformed somatic embryos. The regeneration medium used may be a general growth medium, such as the growth medium described herein, supplemented with the selection agents, and when cocultivation with Agrobacterium was used as a transformation method, the medium will usually include an anti-Agrobacterium antibiotic.

Selection is also possible at the germination/regeneration stage where transformed embryos germinate to regenerate transformed plants on germination/regeneration medium supplemented with a selective agent, while untransformed embryos do not. Early stages of normal embryo germination/regeneration are characterized by hypocotyl elongation, emergence of cotyledonary leaves and chlorophyll development. In late stages of germination/regeneration, cotyledonary or ordinary leaves develop and roots form. Germination/regeneration of somatic embryos and transformed somatic embryos may not be normal, and may require selecting the most normal plantlets from among multiple plantlets arising from abnormal germinated somatic embryos. The regenerated plants thus formed may be cultured for 2 to 10 weeks on germination/regeneration medium before they are ready to be transferred to soil. A plant which is ready to be transferred to soil can be recognized by its containing well formed leaves and roots, and continued emergence of additional well formed leaves as would be expected from a normal plant grown from a seed.

Well developed plantlets can then be transferred to, for example, a greenhouse or elsewhere in a conventional manner for tissue culture plantlets.

Transformation of the resulting plantlets can be confirmed by assaying the plant material for any of the phenotypes which have been introduced by the exogenous DNA. In particular, suitable assays exist for determining the presence of certain reporter genes, such as B-glucuronidase and GFP as described hereinabove. Other procedures, such as PCR, restriction enzyme digestion, Southern blot hybridization, and Northern blot hybridization may also be used.

### Transformation of Crambe Somatic Tissue

In the second transformation method, a preferred material for transformation of somatic tissue leading to transformed undifferentiated callus is hypocotyl tissue, preferably taken from within about 10 mm of the shoot apex of a seedling which was placed on media for germination fewer than seven days previously, preferably placed on media for germination about four days previously. This hypocotyl tissue is preferably sliced transversely into disks about 1 mm in thickness.

In order to achieve the desired transformation, the hypocotyl disks may be subjected to a transformation protocol such as particle gun DNA delivery or cocultivation with an Agrobacterium species carrying the exogenous DNA sequence to be transferred, preferably with an Agrobacterium species carrying the exogenous DNA sequence to be transferred.

For transformation by Agrobacterium cocultivation, incubation is preferably achieved in a cocultivation medium that includes nutrients, an energy source, and an induction compound that is used to induce the virulence (vir) region of Agrobacterium to enhance transformation efficiency. The induction compound can be any phenolic compound which is known to induce such virulence, preferably being acteosyringone (AS) present at from about 10 to 600 μM, preferably at about 100-300 μM. Hypocotyl disks are combined with the Agrobacterium cells in the cocultivation medium at a moderate temperature, typically in the range from about 20° C. to 28° C., preferably at about 22° C.-25° C., from two to four days, and usually for about three days. The medium is preferably kept in the dark, and the cocultivation continued until the Agrobacteria have grown sufficiently so that observable bacterial growth is present. The Agrobacterium cells are initially present at a concentration from about 10⁷ to 10⁹ cells/ml, preferably at about 10⁸ cells/ml. Usually, about 100 to 2000 hypocotyl disks are used in a total culture volume of about 5 to 25 ml. Preferably, the hypocotyl disks and Agrobacterium cells are placed on a filter paper matrix, such as Whatman #1, or glass microfibre filter, on the cocultivation medium during the cocultivation process.

After transformation is completed, the transformed hypocotyl disks are placed on a suitable selection medium including a plant selection agent which permits identification of transformed undifferentiated callus based on the presence of the marker introduced as part of the exogenous DNA.

The transformed cells may be identified or selected and, if desired, regenerated into transformed plants. The methods of the invention do not depend on any particular method for identifying or selecting transformed cells from undifferentiated callus and for regenerating such cells into transformed Crambe plants. Identification methods may involve utilizing a marker gene, such as GFP, or a cell cycle gene, such as CKI, Cyclin D. Methods for using GFP and cell cycle genes are found in U.S. Pat. Nos. 6,300,543, 60/246,349 and 09/398,858 and are incorporated by reference. Selection methods typically involve placing the undifferentiated callus on a medium that contains a selective agent, promotes regeneration or both. If, for example, the nucleotide construct comprises a selectable marker gene for herbicide resistance that is operably linked to a promoter that drives expression in a plant cell, then selection of the transformed cells may be achieved by adding an effective amount of the herbicide to the medium to inhibit the growth of or kill non-transformed cells. Such selectable marker genes and methods of use are well known in the art. Methods and media employed in the regeneration of transformed Crambe plants from transformed cells of undifferentiated callus are described herein. Generally, such methods comprise contacting Crambe undifferentiated callus with a medium containing an effective concentration of an auxin or combination of auxins and an effective concentration of a cytokinin or combination of cytokinins. Any method known in the art for identifying or selecting transformed plant cells and regenerating transformed Crambe plants may be employed in the methods of the present invention. The methods of the invention do not depend on a particular nucleotide construct. Any nucleotide construct that may be introduced into a plant cell may be employed in the methods of the invention. Nucleotide constructs of the invention comprise at least one nucleotide sequence of interest operably linked to a promoter that drives expression in a plant cell. The nucleotide constructs may also comprise identification or selectable marker gene constructs in addition to the nucleotide sequence of interest. This paragraph generally cites U.S. Pat. No. 7,057,089 to Ranch and Marsh.

The selective media is placed in a Petri dish with the hypocotyl disks. The selection medium is a growth and regeneration medium, such as the medium REG which is described in the example section hereinafter, supplemented with the selection agent, and in the case that cocultivation with Agrobacterium was used as a transformation method, usually including an anti-Agrobacterium antibiotic. Again, any antibiotic capable of killing Agrobacterium without harming plant tissues as described herein. Suitable plant selection agents include Geneticin® (1-100 mg/L), Asulam (2-200 mg/L), kanamycin (50-500 mg/L), etc. A preferred concentration of Geneticin is 25 mg/L.

The selection culture is maintained typically for a time sufficient to permit transformed undifferentiated callus to grow and induce the organogenic regeneration of shoots from the undifferentiated callus. Typically, the selection culture will last from about 30 to 60 days, partly depending on the concentration and relative activity of the plant selective agent.

While viability is indicative that the undifferentiated callus and the regenerated shoots originating from the undifferentiated callus produced on the selective medium are transformed, it is usually desirable to confirm transformation using a standard assay procedure, such as Southern blotting, Northern blotting, restriction enzyme digestion, polymerase chain reaction (PCR) assays, or through the use of reporter genes. Suitable reporter genes and assays include glucuronidase (GUS) assays as described by Jefferson, GUS Gene Fusion Systems User's Manual, Cambridge, England (1987) and GFP expression (PCT patent publication WO9741228) These assays can be performed immediately following the transformation procedures or at any subsequent point during the regeneration of the transformed plant materials according to the present invention.

In a preferred embodiment of the invention, the transformed regenerated shoots are then placed on a normalization/growth medium such as MSO that includes plant selection agents in order to produce fully transformed plants. The normalization/growth medium is a general growth medium, such as the one which is described in the examples section hereinafter, supplemented with the selection agents, and in the case that cocultivation with Agrobacterium was used as a transformation method, usually including an anti-Agrobacterium antibiotic.

Well developed plantlets with roots can then be transferred to, for example, a greenhouse or elsewhere in a conventional manner for tissue culture plantlets. Transformation of the resulting plantlets can be confirmed by assaying the plant material for any of the phenotypes which have been introduced by the exogenous DNA. In particular, suitable assays exist for determining the presence of certain reporter genes, such as B-glucuronidase and GFP as described hereinabove. Other procedures, such as PCR, restriction enzyme digestion, Southern blot hybridization, and Northern blot hybridization may also be used.

The methods described above, and in the following examples, are applicable to a number of different Crambe abyssinica varieties, including “Bel Ann”, and “Meyer”. The preferred choice of specific protocols may to some extent be genotype specific. Thus, one skilled in the art can readily adapt the present method to any Crambe abyssinica variety.

### EXAMPLES

It is to be understood that this invention is not limited to the particular devices, machines, materials and methods described. Although particular embodiments are described, equivalent embodiments may be used to practice the invention.

The invention, now being generally described, will be more readily understood by reference to the following examples, which are included merely for purposes of illustration of certain aspects and embodiments of the present invention and are not intended to limit the invention.

### Example I

### Transformation of Crambe Somatic Tissues by Cocultivation with Agrobacterium tumefaciens

Seeds of Crambe abyssinica varieties Meyer and Bel Ann were sterilized by immersion in 20% bleach for 10 minutes followed by three rinses in sterile distilled water. All manipulations after the sterilization steps were performed in an aseptic manner in a laminar air flow cabinet. The sterilized seeds were planted onto solid Basal Medium (MSO) and incubated in the dark at 25° C. for four days, during which time the seeds germinated and produced elongated (etiolated) hypocotyls. Five explants from each hypocotyl were collected by slicing approximately 1 mm thick disks from the region just below the cotyledon attachment point. Care was taken to avoid inclusion of the apical meristem in these explants.

The explants were cocultivated with Agrobacterium strain LSLJ4571 by soaking them in a 10-fold dilution of a culture grown in Agrobacterium Growth Medium (MinA) to saturation. Agrobacterium strain LSLJ4571 is strain LBA4404 (Hoekema) which carries the binary plasmid pSLJ4571 (35SNPT/35SGUS). The explants were then transferred onto a filter paper disks which overlaid Regeneration Medium (REG) supplemented with 100 UV Acetosyringone, and allowed to cocultivate for three days at 25° C. in darkness. These cocultivated explants were then transferred to REG medium lacking acteosyringone but supplemented with 25 or 50 mg/L Geneticin® (Phytotechnology Laboratories, Shawnee Mission, Kans.; concentrations of 1-100 mg/L, e.g., 5, 10, 15, 25, 30, 35, 40, 45, 50, 75, or 100 mg/L, or intermediate concentrations, would also be suitable) and 150 mg/L Timenton (Phytotechnology Laboratories; concentrations of 50 mg/L-150 mg/L or more would also be suitable) and incubated at 25° C. under white light with 12 hour photoperiods provided by cool white bulbs. Incubation in these conditions was continued with transfer to fresh media of the same composition every 2-3 weeks until transformed calli with shoots formed. The transformed shoots were then transferred onto solidified basal medium for growth and rooting. If rooting does not occur spontaneously, the shoots can be treated with an auxin-containing medium such as an indole 3-butyric acid (IBA)-containing medium to induce them to root. Rooted shoots were transplanted into soil and grown to maturity. The transformation efficiency of this process was greater than 1%, and transformation efficiencies of at least 2%, or at least 5%, or at least 10%, or at least 15%, or at least 20%, or at least 25%, can be produced.

### Example II

### Improvement of Regeneration of Crambe Somatic Tissue by Selection for Improved Genetic Sub-Populations

Etiolated hypocotyls from 91 Crambe seeds of the variety Meyer were prepared as described in Example I. Each of these hypocotyls was individually assayed for its relative capacity for regeneration by cutting into about 20 segments and transfer to REG medium. The number of regenerating shoots from each hypocotyl was recorded. The apical meristems from some of these seedlings were grown into mature plants by transfer to MSO medium, and when rooted, transfer to soil and growth into mature plants. These mature plants were self-pollinated and the resulting seeds were collected. A numbering system was used such that the seeds from each mature plant and the original hypocotyl associated with it could be identified. The hypocotyl regeneration assays showed that 61 of the 91 (67%) hypocotyls from Meyer seedlings have little or no capacity for regeneration, whereas only 13 (14%) of these hypocotyls regenerated reasonably well (as defined by more than 5 shoots emerging from callus produced on the 20 assayed hypocotyl disks).

The heritability of this increased capacity for regeneration was demonstrated by assaying 11 of the lines of seeds produced on the plants which had been assayed for regenerability as described above. There was a very high degree of correlation between the hypocotyl regenerability of the parent and the hypocotyl regenerability of the selfed seed produced, with the parent produced from the meristem attached to the most highly regenerable hypocotyl (#71) producing the most regenerable seedling hypocotyls, and the parents produced from meristems attached to non-regenerable hypocotyls producing non-regenerable or poorly regenerable seedling hypocotyls. Parents produced from meristems attached to hypocotyls with intermediate regenerability generally produced seedling hypocotyls with intermediate regenerability.

This example is a demonstration of a method for producing seed lines (e.g. Line #71) with hypocotyls having increased regenerability relative to control seed lines (e.g., parental lines, or lines of plants not selected for increased regenerability), that is, the hypocotyl tissue is derived from a plant line selected for greater hypocotyl regenerability than that of a control plant line. The method can be repeated, for example, by using selfed seed lines with increased hypocotyl regenerability as starting material. Each iteration of the method, that is, each generation and selection for regenerability, would result in seed lines with further increases in hypocotyl regenerability relative to a parental or control plant.

### Example III

### Improved Transformation of Crambe Somatic Tissue Using Genetic Sub-Populations Selected for Improved Regeneration

Seedlings produced as a result of self-pollination of plant #71 described in Example II were used in a transformation experiment according to the protocol of Example I. In the current example, hypocotyls from 100 seedlings from Line #71 were cocultivated. Twenty four transformed calli were produced and 10 of them regenerated shoots. All calli and regenerated shoots expressed the GUS gene, indicating that no escapes were produced. Previous experiments using hypocotyls from non-selected Meyer seedlings produced transformed calli at similar frequencies, but in these experiments, two or fewer transformed shoots were produced. No escapes were produced in these previous experiments.

### Example IV

### Transformation of Crambe Embryogenic Callus by Cocultivation with Agrobacterium tumefaciens

Crambe seeds of the varieties Meyer and Bel Ann were planted in soil, and grown to maturity in soil. Water and fertilizer were provided when needed with a solution of 0.4 g/L of Peters fertilizer at each watering. Plants were grown at 25° C. under continuous white light provided by 400 Watt HID fixtures (Voigt Lighting Industries, Inc). Approximately 6-8 weeks after planting, the plants had flowered, and clusters of immature flower buds were harvested. The clusters of immature flower buds were sterilized by immersion into approximately 100 ml of a 10% solution of bleach with one drop of Triton X-100 added and swirled for 10 minutes, and rinsed twice with sterile distilled water. All manipulations after the above sterilization steps were done using aseptic methods in a laminar air flow cabinet. The immature flower buds which were less than 1.5 mm in diameter were used as a source from which to obtain immature anthers. The immature anthers were micro-dissected from the flowers and transferred to plates of solid ECIGM media. These plates were incubated at 25° C. in white light provided by cool white florescent tubes with a day length of 12 hours. The embryogenic callus (ecallus) that developed was transferred to fresh solid ECIGM media and replaced in the same conditions as for initiation. Ecalli proliferated into larger masses of ecallus in these conditions, and these ecalli were subcultured by transfer of small (1-2 mm) pieces to fresh solid ECIGM media every 2-3 weeks.

Transformation of the ecallus described above was initiated by cocultivation with Agrobacterium tumefaciens. A mass of about 600 mg of ecallus was transferred into five ml of MinA media, and (500 μl) of the Agrobacterium tumefaciens strain LSLJ4571, which had been grown to saturation in MinA medium was added. Agrobacterium strain LSLJ4571 is strain LBA4404 (ref) which carries the binary plasmid pSLJ4571. The ecallus and bacterial cells were thoroughly mixed, and the inoculated ecalli were then transferred onto ECIGM medium supplemented with 100 μM acteosyringone and cocultivated at 22° C. in the dark for two days.

Selection for transformed Crambe cells and counterselection against the Agrobacterium was performed by transferring the cocultivated ecalli onto solid ECM medium supplemented with 25 mg/L Geneticin for selection and 150 mg/L Timenton for counterselection. The ecalli were incubated in the same culture conditions as were indicated above for ecallus initiation and growth. These selection/counterselection cultures were transferred to fresh medium every 2-3 weeks. Following 25 days of incubation, most of the ecalli were brown, or black, and appeared dead, but a minority of the ecalli had segments that appeared to be growing and thus resistant to the Geneticin. A GUS assay demonstrated the presence of the GUS gene in some or all of the tissues assayed in all nine of these ecallus lines. Several of the Geneticin-resistant lines were transferred to MSO medium to induce regeneration of whole plants, and rooted, transformed plants derived from varieties Meyer and Bel Ann were produced. One of the transformed Meyer plants was allowed to mature, flower, self-pollinate and set seeds. When mature, these progeny seeds were collected, germinated, and the seedlings assayed for the presence of the GUS gene. Seventeen of the twenty three seedlings assayed stained a dark blue color indicating the presence of a functional GUS gene. These data fit a Mendelian segregation ratio of 3 GUS positive: 1 GUS negative and demonstrate that the transformant was a non-chimeric transformed plant with the transgene stably integrated at a single locus, and heritable via the gametes from one generation to the next.

### Example V

### Transformation of Crambe Embryogenic Callus by Particle Bombardment

Transformation of the ecallus may also be accomplished by bombardment of the embryogenic callus by microparticles coated with a nucleic acid construct (i.e., a plasmid, vector, cassette or other DNA preparation) of choice. The PDS-1000 Biolistics particle bombardment device may be used to deliver DNA to the target cells. The operation of this device is detailed in the operating instructions available from the manufacturer (Bio-Rad Laboratories, Hercules, Calif.).

Briefly, DNA and particles of materials with large specific gravity (e.g., tungsten, gold, palladium, or platinum) are associated and the preparation is dried on plastic macrocarriers. Prior to association with the transforming DNA, tungsten particles are prepared essentially as described in U.S. Pat. No. 5,990,387 to by Tomes et al. Such particles are also known as microparticles or microprojectiles. Prior to each bombardment, the expendables are mounted in the device. Expendables include the macrocarrier with a dried DNA/particle preparation, a rupture disk, and a stopping screen. The material intended to be bombarded is positioned upon on target platform. The embryogenic callus may be pre-treated prior to introduction into the particle gun by contacting it with an osmotic conditioning agent. An osmotic conditioning agent or osmoticum may be beneficial to particle gun mediated transformation. While the precise mechanism has not been identified, a preferred explanation holds that plasmolyzed cells, a consequence of an osmotic conditioning treatment, are less apt to lyse when penetrated by a particle.

Next, the chamber of the device is evacuated with a vacuum pump to near 28 mm Hg. A small reservoir behind the rupture disk is then slowly filled with helium. When the helium pressure in this chamber rises sufficiently, the rupture disk breaks and releases a burst of helium. The helium burst pushes against the macrocarrier and accelerates it towards the stopping screen. The stopping screen, a metal mesh, abruptly stops the macrocarrier. The DNA/particle preparation that is dried upon the macrocarrier is released from the macrocarrier and continues on a path to strike the target. The chamber is equalized with the atmosphere, and the expendibles are removed. The same or a different osmotic agent may be used as a post-particle gun transformation treatment. This example generally cites U.S. Pat. No. 7,057,089 to Ranch and Marsh as to methods for operation of the PDS-1000 particle bombardment device.

Following DNA introduction into embryogenic callus cells by particle gun treatment, the embryogenic callus is cultured in the presence of a selective agent to identify and/or select for transformed cells, and the transformed cells are grown and regenerated into whole plants as is described in Example IV.

### Example VI

### Transformation of Crambe Somatic Tissues by Cocultivation with an Agrobacterium tumefaciens Strain Engineered to Confer the Ability to Produce Wax Esters on Transgenic Seeds

Seeds of Crambe abyssinica are germinated and hypocotyl explants taken, these explants are cocultivated, selected, and transgenic plants are recovered as in Example I, except that the Agrobacterium strain used for cocultivation is not LSLJ4571, but rather an LBA4404 Agrobacterium strain containing a binary plasmid carrying a KCS gene, a fatty acyl-CoA reductase gene, a wax synthase gene, and a plant selectable marker gene, each operably linked to an appropriate promoter, such as are present on the plasmid pCGN8559 (described in Lassner et al. 1999).

The explants were cocultivated with Agrobacterium strain LSLJ4571 by soaking them in a 10-fold dilution of a culture grown in Agrobacterium Growth Medium (MinA) to saturation. Agrobacterium strain LSLJ4571 is strain LBA4404 (Hoekma, 1983) which carries the binary plasmid pSLJ4571. pSLJ4571 carries an

NPTII gene operably linked to a 35S promoter and a GUS gene operably linked to a ³⁵S promoter. The Agrobacterium strain LSLJ4571 was a gift from Jonathan Jones at the Sainsbury Laboratory of the John Innes Centre in Norwich, UK.

### Formulations for Media Described Above

The following formulations are for liquid media. If solid media are required, 2.5 g/L Gelrite® are added before autoclaving.

### Basal Medium (MSO)

MS salts mixture (1×)

MS vitamin mixture (1×)

2-(N-Morpholino)ethanesulfonic Acid, Free Acid (0.6 g/L)

NZ amine (0.5 g/L)

Glucose (10 g/L)

pH adjusted to 5.7

Agrobacterium growth medium (MinA)

Sucrose (2 g/L)

1 g/L Ammonium Sulfate

0.5 g/L Sodium Citrate

13.7 g/L Potassium Hydrogen Phosphate

4.5 g/L Potassium Phosphate, monobasic

120.37 g/L Magnesium Sulfate

Embryogenic callus induction and growth medium (ECIGM)

MSO Medium to which is added:

Kinetin (0.5 mg/L)

2,4-D (0.5 mg/L)

Regeneration medium (REG)

Basal medium

0.5 mg/L TDZ

0.5 mg/L NAA

### REFERENCES CITED

Ainley et al. (1993) Plant Mol. Biol. 22: 13-23
An et al. (1988) Plant Physiol. 88: 547-552
Ausubel et al. (1997-2001; eds.) Current Protocols in Molecular Biology, John Wiley & Sons (1997 and supplements through 2001
Baerson et al. (1993) Plant Mol. Biol. 22: 255-267
Baerson et al. (1994) Plant Mol. Biol. 26: 1947-1959
Bairn et al. (1991) Proc. Natl. Acad. Sci. USA 88:5072-5076
Barkley et al. (1980) in The Operon, pp. 177-220
Baumann et al., (1999) Plant Cell 11: 323-334
Berger and Kimmel (1987), “Guide to Molecular Cloning Techniques”, in Methods in Enzymology, vol. 152, Academic Press, Inc., San Diego, Calif.
Bevan (1984) Nucleic Acids Res. 12: 8711-8721
Bird et al. (1988) Plant Mol. Biol. 11: 651-662
Bonin (1993) Ph.D. Thesis, University of Heidelberg
Brown et al. (1987) Cell 49: 603-612
Buchel et al. (1999) Plant Mol. Biol. 40: 387-396
Christopherson et al. (1992) Proc. Natl. Acad. Sci. USA 89:6314 6318
Daly et al. (2001) Plant Physiol. 127: 1328-1333
Degenkolb et al. (1991) Antimicrob. Agents Chemother. 35:1591-1595
Deuschle et al. (1989) Proc. Natl. Acad. Sci. USA 86:5400-5404
Deuschle et al. (1990) Science 248:480-483
Endres and Schatz (1991) Crambe Production Practices, Bulletin A-1010. Fargo: North Dakota State University Extension Service
Erickson and Bassin (1990) Bulletin 656: Agricultural Experiment Station (July, 1990), Kansas State University, Manhattan, Kans.
Figge et al. (1988) Cell 52:713-722
Fromm et al. (1989) Plant Cell 1: 977-984
Fuerst et al. (1989) Proc. Natl. Acad. Sci. USA 86: 2549-2553
Gan and Amasino (1995) Science 270: 1986-1988
Gao et al. (1998) Acta Botanica Yunnanica 20: 247-250 (China)
Gardner et al. 1991. Field Scale-Evaluation of Crambe as an Alternative Crop, Progress Report. Fargo: North Dakota State University
Gatz (1997) Annu. Rev. Plant Physiol. Plant Mol. Biol. 48: 89-108
Gelvin et al. (1990) Plant Molecular Biology Manual, Kluwer Academic Publishers
Gill et al. (1988) Nature 334:721-724
Gossen et al. (1992) Proc. Natl. Acad. Sci. USA 89:5547-5551
Gossen (1993) Ph.D. Thesis, University of Heidelberg
Guevara-Garcia (1998) Plant Mol. Biol. 38: 743-753
Herrera-Estrella et al. (1983) Nature 303: 209
Hillenand-Wissman (1989) Topics Mol. Struc. Biol. 10:143-162
Hlavka et al. (1985) Handbook of Experimental Pharmacology, Vol. 78 (Springer-Verlag, Berlin)
Hoekema et al. (1983) Nature 303: 179-181
Hu et al. (1987) Cell 48:555-566
Jones (1988) Monograph British Plant Growth Regulator Group (17): 37-55
Kaiser et al. (1995) Plant Mol. Biol. 28: 231-243
Klee (1985) Bio/Technology 3: 637-642
Kleinschnidt et al. (1988) Biochemistry 27:1094-1104
Ku et al. (2000) Proc. Natl. Acad. Sci. USA 97: 9121-9126
Kuhlemeier et al. (1989) Plant Cell 1: 471-478
Labow et al. (1990) Mol. Cell. Biol. 10:3343-3356
Lassner et al. (1999) Producing Wax Esters in Transgenic Plants by Expression of Genes Derived from Jojoba. P. 220-224. In: Perspectives on new crops and new uses. J. Janick (ed.), ASHS Press, Alexandria, Va.
Lear et al. (1989) Crambe Production, Factsheet. Lincoln: Cooperative Extension Service, University of Nebraska
Manners et al. (1998) Plant Mol. Biol. 38: 1071-1080
Minor. Crambe Production Practices. Unpublished. Columbia: University of Missouri.
Nebraska Cooperative Extension (1991) Nebraska Spring Wheat, Oats, Barley, Canola and Crambe Variety Tests 1991, Ec 91-102-C. Lincoln: University of Nebraska Cooperative Extension Service
Odell et al. (1985) Nature 313: 810-812
Odell et al. (1994) Plant Physiol. 106: 447-458
Ohl et al. (1990) Plant Cell 2: 837-848
Oliva et al. (1992) Antimicrob. Agents Chemother. 36:913-919
Reines et al. (1993) Proc. Natl. Acad. Sci. USA 90:1917-1921
Reznikoff (1992) Mol. Microbiol. 6:2419-2422
Rieger et al. (1976) Glossary of Genetics and Cytogenetics: Classical and Molecular, 4th ed., Springer Verlag, Berlin
Ringli and Keller (1998) Plant Mol. Biol. 37: 977-988
Sambrook et al. (1989) Molecular Cloning: A Laboratory Manual, 2nd Ed., Cold Spring Harbor Laboratory, Cold Spring Harbor, N.Y.
Schaffner and Sheen (1991) Plant Cell 3: 997-1012
Sell, et al. (1992) Crambe as a Specialty Crop in North Dakota, Agricultural Economics Report No. 268. Fargo: North Dakota State University, Department of Agricultural Economics
Siebertz et al. (1989) Plant Cell 1: 961-968
Shi et al. (1998) Plant Mol. Biol. 38: 1053-1060
Sonntag and Rudloff (2001) Cruciferae Newsletter 23: 33-34
Sonntag and Gramenz (2004) Cruciferae Newsletter 25: 101-102
Tudge (2000) in The Variety of Life, Oxford University Press, New York, N.Y. pp. 547-606
van der Kop et al. (1999) Plant Mol. Biol. 39: 979-990
Van Dyne et al. (1990) Industrial Feedstocks and Products from High Erucic Acid Oil: Crambe and Industrial Rapeseed. Columbia: University of Missouri, 1990.
Weissbach and Weissbach (1989) Methods for Plant Molecular Biology, Academic Press
Willmott et al. (1998) Plant Molec. Biol. 38: 817-825
Wyborski et al. (1991) Nucleic Acids Res. 19:4647-4653
Yarranton (1992) Curr. Opin. Biotech. 3:506-511
Yao et al. (1992) Cell 71:63-72
Zambretti et al. (1992) Proc. Natl. Acad. Sci. USA 89:3952-3956

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the appended claims. Modifications that become apparent from the foregoing description fall within the scope of the claims.

1. A method for producing a transformed plant of the genus Crambe, wherein a polynucleotide of interest is integrated into a cell of the transformed plant in a stable manner, and the transformed plant is able to transmit at least one transgene to a progeny plant, said method comprising the steps of:
(a) generating an embryogenic callus from a target plant of the genus Crambe;
(b) transforming the embryogenic callus with the polynucleotide of interest and a selection marker;
(c) selecting for a transformed embryogenic callus with a selective agent at a concentration that prevents a chimeric plant composed of transformed cells and non-transformed cells from being regenerated from the embryogenic callus; and
(d) producing the transformed plant from the transformed embryogenic callus.

2. The method of claim 1, wherein the selective marker is a polynucleotide that encodes neomycin phosphotransferase II.

3. The method of claim 2, wherein the concentration of the selective agent is 5 to 100 mg/L.

4. The method of claim 1, wherein the polynucleotide of interest is introduced into the embryogenic callus with an Agrobacterium cocultivation method or with a biolistic transformation method.

5. The method of claim 1, wherein the target plant is a Crambe abyssinica variety other than Galactica.

6. The method of claim 1, wherein the transformed plant is comprised entirely of transformed cells.

7. A transformed Crambe plant produced by the method of claim 1.

8. A method for producing a transformed plant of the genus Crambe, wherein a polynucleotide of interest is integrated into a cell of the transformed plant in a stable manner, and the transformed plant is able to transmit at least one transgene to a progeny plant, said method comprising the steps of:
(a) collecting an explant from the target plant;
(b) transforming the explant with the polynucleotide of interest and a selection marker;
(c) selecting for a transformed explant with a selective agent at a concentration that prevents a chimeric plant composed of transformed cells and non-transformed cells from being regenerated from the transformed explant; and
(d) producing the transformed plant from the transformed explant.

9. The method of claim 8, wherein the selective marker is a polynucleotide that encodes neomycin phosphotransferase II.

10. The method of claim 9, wherein the concentration of the selective agent is 5 to 100 mg/L.

11. The method of claim 8, wherein the polynucleotide of interest is introduced into the explant with an Agrobacterium cocultivation method or with a biolistic transformation method.

12. The method of claim 8, wherein the target plant is a Crambe abyssinica variety other than Galactica.

13. The method of claim 8, wherein the transformed plant is comprised entirely of transformed cells.

14. The method of claim 8, wherein the explant is derived from seedling tissue of the target plant.

15. The method of claim 8, wherein the explant is from hypocotyl tissue of the target plant.

16. The method of claim 8, wherein the target plant is selected for having greater regenerability than a control plant line.

17. The method of claim 16, wherein the target plant is selected for having greater hypocotyl regenerability than a control plant line.

18. A transformed Crambe plant produced by the method of claim 8.

19. A plant of the genus Crambe having greater regenerability than a control plant, wherein the plant is selected from a plurality of Crambe plants on the basis of having greater regenerability than the control plant.

20. The plant of claim 19, wherein the plant of the genus Crambe has greater hypocotyl regenerability than the control plant.

21. Hypocotyl tissue derived from the plant of claim 20.

22. A method for producing a plant of the genus Crambe having greater regenerability than a control plant, the method steps comprising:
(a) germinating a seed of the genus Crambe;
(b) removing a hypocotyl from the germinating seed, or optionally separating the hypocotyl into hypocotyl segments.
(c) transferring the hypocotyl or hypocotyl segments to a medium and an environment that supports regeneration of the hypocotyl or hypocotyl segments;
(d) selecting a specific hypocotyl or a specific hypocotyl segment that produces a higher number of shoots than the average number of shoots produced by the hypocotyl or hypocotyl segments;
(e) growing an apical meristem or a regenerated shoot from the specific hypocotyl or the specific hypocotyl segment of step (d) into a mature plant;
(f) self-pollinating the mature plant or pollinating the mature plant with pollen of another plant that has greater hypocotyl regenerability than the control plant, and collecting a resulting progeny seed; and
(h) growing a progeny plant from the progeny seed, wherein the progeny plant has greater hypocotyl regenerability than the control plant.
